(19) United States
(12) Patent Application Publication
(10) Pub. No.: US 2015/0173664 A1
(43) Pub. Date: Jun. 25, 2015

(54) METHOD, DEVICE AND KIT TO IMPROVE BLOOD SAMPLES SIZE FROM LANCET OR NEEDLE

(71) Applicants: Richard C. FUISZ, Beverly Hills, CA (US); Joseph M. FUISZ, Surfside, FL (US)

(72) Inventors: Richard C. FUISZ, Beverly Hills, CA (US); Joseph M. FUISZ, Surfside, FL (US)

(21) Appl. No.: 14/135,831

(22) Filed: Dec. 20, 2013

(51) Int. Cl. A61B 5/151; A61B 5/15

(52) U.S. Cl. CPC A61B 5/15146; A61B 5/150343; A61B 5/150061

(57) ABSTRACT

A collection device holder is designed to hold a plurality of blood collection tubes. The holder includes a support member and a plurality of holding portions. Each of the plurality of holding portions is configured to hold a blood collection tube with respect to the support member such that inlets of the plurality of blood collection tubes are arranged in a predetermined pattern with respect to one another. In a preferred aspect, the predetermined pattern corresponds to a predetermined pattern of a plurality of spaced sharpened tips of a lancet device.

### BACKGROUND OF THE INVENTION

Taking small blood samples from conventional lancet or needle pricks is well known both to the patent art and daily life. The use of personal, handheld glucose meters is the most common example from daily life. Many companies, including large diagnostic companies like Abbott and Bayer, market handheld glucose meters that rely on conventional finger prick blood samples to render presumptively accurate glucose readings. The convenience of these devices is an essential utility for the quality of life of millions of Americans who suffer from diabetes.

Seemingly like all electronic devices, there is a pronounced trend towards smaller, more powerful devices requiring smaller volumes of blood. Advances in the microfluidic handling of blood samples as well as analysis methods (be it reagent based chemistries, spectroscopy and the like) have brought us to the cusp of an age where blood analyzers relying on small blood samples can perform many valuable tests.

For example, the EPOC® system marketed by Alere is a handheld point of care blood analysis tool that can test for nine analytes (pH, pCO2, pO2, Na+, K+, Ca++, Glu, Hct, Lac) using one test card and a single blood sample (92 μL). The sample may be drawn from capillary source (i.e. a finger prick). While the EPOC® system is directed to use in care settings (see e.g. http://www.accessdata.fda.gov/cdrh_docs/reviews/K092849.pdf), one can imagine the transitioning of the EPOC® system or a future, related product embodiment for home use.

Theranos (www.theranos.com) claims to be launching a system that will employ “tiny finger stick or collect a micro-sample from a venous draw.” Theranos does not specify the collection sample, but it appears from the blood sample diagram on the Theranos website (http://www.theranos.com/our-technology) that their sample is approximately 0.18 ml (nota bene, this is merely a visual estimate from the website; the inventors cannot identify a specific volume on the Theranos website. Accordingly the inventors do not know the actual target sample size for this system).

Now, the Theranos system is initially to be deployed in Walgreens with trained phlebotomists to oversee sample collection. Now this begs a number of questions: can a phlebotomist reliably obtain an 0.18 ml sample (or larger) from a finger prick? Could a patient reliably obtain an 0.18 ml sample (or larger) from him or herself in a home setting? How about a larger sample for additional tests?

It seems there is real question as to the ability to reliably obtain such larger sample sizes from a fingerprick. Theranos suggests that their sample could be collected by fingerprick or traditional venous draw: “our certified phlebotomists can use a tiny finger stick or a micro-sample from a venous draw.”

### SUMMARY OF THE INVENTION

One aspect of the present invention relates to a collection device holder for a plurality of blood collection tubes, the holder comprising a support member and a plurality of holding portions, each of the plurality of holding portions being configured to hold a blood collection tube with respect to the support member such that inlets of the plurality of blood collection tubes are arranged in a predetermined pattern with respect to one another.

Another aspect of the present invention relates to a collection device holder for a holding at least one blood collection tube, comprising a holder comprising a support member and a at least one holding portion configured to hold at least one blood collection tube, and a device for creating a lower pressure region in the capillary tube.

Another aspect of the present invention relates to a lancet device comprising a body portion and a sharpened tip portion, the sharpened tip portion having an anti-clotting agent provided thereon.

Another aspect of the present invention relates to a lancet device comprising a body barrel portion and a plurality of spaced sharpened tip portions, each of the plurality of spaced sharpened tip portions having a thickness of 21-33 gauge.

Yet another aspect of the present invention relates to a lancet device comprising a body portion and a plurality of spaced sharpened tip portions, each of the plurality of spaced sharpened tip portions being spaced from one another close enough to cause coalescence of blood from multiple punctures in skin of a mammal made by the plurality of spaced sharpened tip portions into a single droplet.

Another aspect of the present invention relates to a kit comprising a lancet device comprising a body portion and a supporting portion for supporting a plurality of spaced sharpened tips in a predetermined pattern with respect to one another; and a collection device holder for a plurality of blood collection tubes, the holder comprising a support member and a plurality of holding portions, each of the plurality of holding portions being configured to hold a blood collection tube with respect to the support member such that inlets of the plurality of blood collection tubes are arranged in a predetermined pattern with respect to one another corresponding to the a predetermined pattern of the plurality of spaced sharpened tips.

Yet another aspect of the present invention relates to a method for collecting blood, comprising producing multiple punctures in skin of a mammal using a lancet device comprising a body portion and a supporting portion supporting a plurality of spaced sharpened tips in a predetermined pattern with respect to one another; providing a collection device holder for a plurality of blood collection tubes, the holder comprising a support member and a plurality of holding portions, each of the plurality of holding portions holding a blood collection tube with respect to the support member such that inlets of the plurality of blood collection tubes are arranged in a predetermined pattern with respect to one another corresponding to the a predetermined pattern of the plurality of spaced sharpened tips; and collecting blood from the multiple punctures by touching the inlets of the plurality of blood collection tubes to the multiple punctures.

Another aspect of the present invention relates to a method for collecting blood using cold to warm cycling to refresh the capillary field prior to lancet use, comprising cooling a portion of the skin of a mammal; then, within 60 seconds after the cooling, warming the portion of the skin; then, within 5 minutes after the warming, producing at least one puncture in skin of a mammal; and collecting blood from the at least one puncture.

Yet another aspect of the present invention relates to a method for collecting blood, comprising producing at least one puncture in skin of a mammal; and collecting blood from the at least one puncture in a collection tube open at each end while creating a pressure differential over the end of the collection tube opposite the collection end so that blood is more easily drawn into the collection tube.

### BRIEF DESCRIPTION OF THE DRAWINGS

FIG. 1 is a perspective view of one embodiment of a lancet device according to the present invention.

FIGS. 2A-2D are schematic end views of lancet devices of the present invention showing one, two, three and four sharpened tip portions, respectively.

FIG. 3A is a perspective view of one embodiment of a collection device holder according to the present invention.

FIGS. 3B-3D are schematic end views of collection device holders of the present invention showing two, three and four holding portions, respectively.

FIGS. 4A and 4B are schematic views of collection device holders of the present invention having devices for creating a lower pressure region in the collection tubes.

### DETAILED DESCRIPTION OF THE INVENTION

It is apparent to the inventors that an easier method and device is required to reliably produce sufficient blood draws from a finger prick (or similar minimally invasive location) so as to enable the use of modern analyzers (and the analyzers of the future) in the home by actual patients (as opposed to trained or certified phlebotomists). Moreover, even where a trained phlebotomist is available, a reliable method is used so that capillary draws will routinely provide sufficient blood volume so as to minimize the availability differences between capillary and venous draw, or attempting to reliably calibrate for such differences.

This invention relates to the use of novel, multi-prick lancets to increase blood draw from a lancet prick. This invention relates to novel collection devices that can efficiently and effectively collect blood released by a multi-prick lancet. This invention relates to the use of a lancet (conventional or multi-prick) that is coated with an anti-clotting agent to enhance the volume of blood available from a lancet-prick.

The multi-prick lancet comprises multiple puncture sites from a single lancet barrel. These multi-prick heads are preferably tightly and precisely spaced to as to allow for the simultaneous multi-puncture of a single finger. However, in order to utilize this system and obtain a greater blood volume, the system includes a holder for the multiple collection tubes which exactly mimics the geographical location of the lancets. This enables all the tubes to be filled simultaneously, before clotting or running of the blood. It also mitigates the need for excessive milking, which can lead to anomalous analyte results in testing. Furthermore, the collection tube holder has a friction effect on the individual tubes to hold them in place vertically and also allow for any depth adjustment on any of the tubes to facilitate collection.

The lancet device of the present invention can utilize lancet devices known in the art for collection of capillary blood. These may include, for example, puncture devices and incision devices that puncture the skin by inserting either a needle or blade into the tissue. The lancet device may include a body portion and a sharpened tip portion, e.g., a needle point or blade.

The multi-prick lancet may comprise two to twenty-five puncture sites, preferably three to six puncture sites. The tips of the multi-prick lancets may have, for example but not limited to, a thickness of 21-33 gauge. The nerve fibers in the finger and skin cannot differentiate clearly between one prick and multiprick as long as the multipricks are in proximity, not separated by more than approximately 15 mm. The lancets can be present in a single barrel which will enable great ease of selection of the multiple lancets and their use as well although it should not be viewed as a limitation and can in fact be multiple mini barrels.

It may be desirable to calibrate the length of the multi-prick heads such that the prick sites have differing effect lengths. This will allow more uniform puncture depth where the pricks are spaced to correspond to the natural curvature of the finger.

The lancet device of the present invention is a modification of lancing devices having any form known in the art. For example, the lancet can be a single use, disposable lancet modified to have an anti-clotting agent applied to the tip and/or modified to have a plurality of tips. Alternatively, a standard reusable lancing device instrument that can be equipped with a lancet can be used with a lancet having an anti-clotting agent applied to the tip or can be modified to accept a plurality of lancets. Such a reusable lancing device instrument can have preset or customizable depth settings for different skin types and can use a manual plunger or a spring-loaded type of plunger as is known in the art.

Rose thorns may also be used as an inexpensive and very effective lancet.

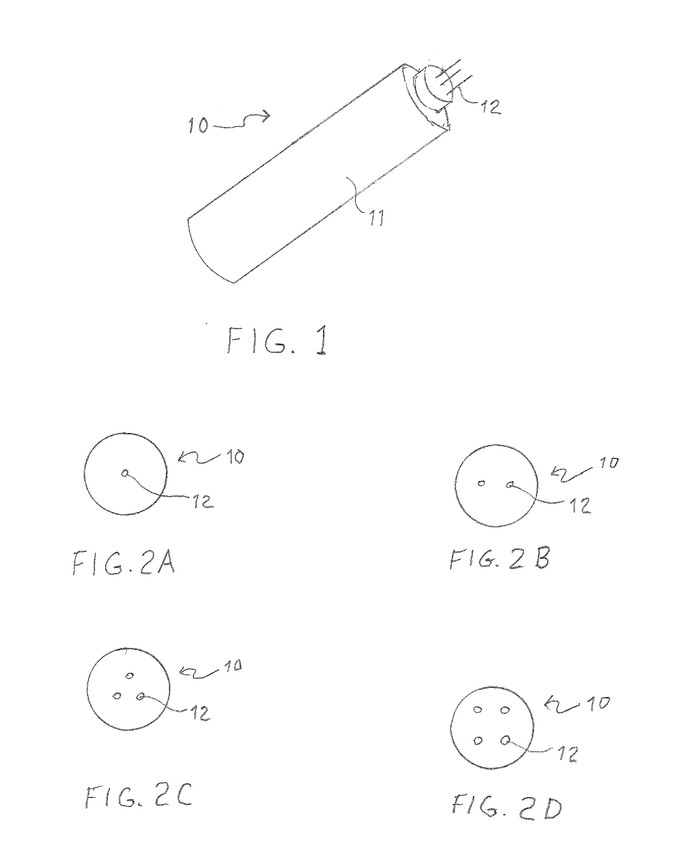

FIG. 1 is a perspective view of one embodiment of a lancet device 10 according to the present invention. As shown in FIG. 1, the lancet device 10 includes a body barrel portion 11 and, in this embodiment, a plurality (three in this embodiment) of spaced sharpened tip portions 12. FIGS. 2A-2D are schematic end views of lancet devices 10 of the present invention showing one, two, three and four sharpened tip portions 12, respectively. As noted above, the present invention is not limited to lancet devices having 1-4 sharpened tip portions; rather, in the case of a multi-prick lancet, the lancet device may comprise two to twenty-five puncture sites.

In the case of the lancet device 10 shown in FIG. 2A having a single sharpened tip portion 12, an anti-clotting agent is applied thereto. In a preferred aspect of the invention in connection with the lancet devices 10 shown in FIGS. 2B-2D, the sharpened tip portions 12 may have an anti-clotting agent applied thereto.

The multiple tip lancets shown in FIGS. 2B-2D are advantageously used with a holder for multiple capillary type tubes with geographic unity. In that case, the tips of the multi-prick lancets may have, for example but not limited to, a thickness of 21-33 gauge. The tips 12 are, in that case, preferably spaced so that separate blood drops can be collected by the multiple capillary type tubes. The particular geographic arrangement of the tip portions 12 is not limited to the embodiments shown in the figures. The multiple tip lancets shown in FIGS. 2B-2D may alternatively be used as to form a larger coalesced blood sample which can be collected by a single, e.g., larger collection tube. In that case, the tips 12 are more closely spaced a larger coalesced blood sample.

Thus, multiple tubes may be used simultaneously to collect the multiple prick sites resulting from a multi-prick lancet. For this use, we contemplate a collection device holder for multiple blood collection vessels (or tubes), e.g., standard capillary blood collection tubes, heparinized or not, which geographically exactly mimics the placement of a multiple head lancet device.

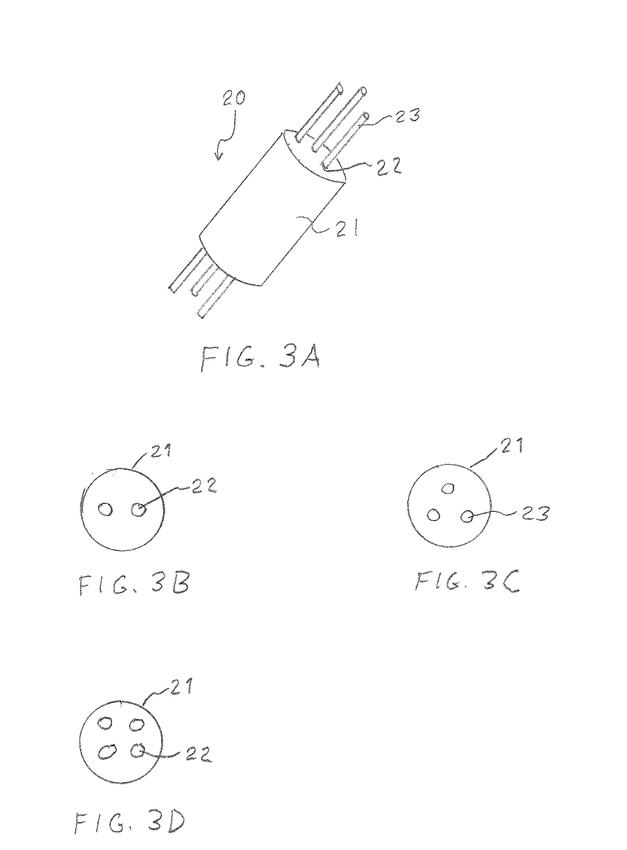

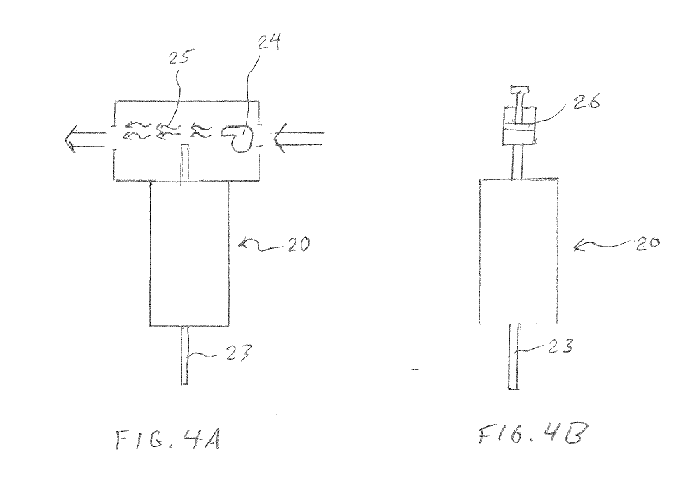

FIG. 3A is a perspective view of one embodiment of a collection device holder 20 according to the present invention. The collection device holder 20 functions to hold a plurality of blood collection tubes (three in the embodiment shown in FIG. 3A). The holder 20 includes a support member 21 and a plurality of holding portions 22, each of the plurality of holding portions being configured to hold a blood collection tube 23 with respect to the support member 21 such that inlets of the plurality of blood collection tubes 23 are arranged in a predetermined pattern with respect to one another. The particular geographic arrangement of the tip portions 12 is not limited to the embodiments shown in the figures. In this embodiment, the collection tube holder 21 has bores which function as the holding portions 22. The bores in this embodiment correspond to an outside diameter of the blood collection tubes 23 so that a friction effect is achieved on the individual tubes 23 to hold them in place vertically and also allow for any depth adjustment on any of the tubes to facilitate collection. For example, in this embodiment, the collection tubes have collection ends not on the same plane to correspond to curvature of, e.g., a fingertip.

FIGS. 3B-3D are schematic end views of collection device holders 20 of the present invention showing two, three and four holding portions 22, respectively. When used in connection with multi-prick lancet devices, the arrangement of holding portions 22 is such that the collection tubes 23 can be arranged in a predetermined pattern with respect to one another corresponding to the a predetermined pattern of the plurality of spaced sharpened tips of the multi-prick lancet devices. For example, the two, three and four holding portions 22, respectively, shown in FIGS. 3B-3D are arranged such that the collection tubes 23 inserted in the holding portions corresponding to the a predetermined pattern of the plurality of spaced sharpened tips of the multi-prick lancet devices of FIGS. 2B-2D, respectively.

Of course the present invention is not limited to the particular friction-type of collection device holder 20 shown in FIGS. 3A-3D. Any means for holding a plurality of blood collection tubes including a support member and a plurality of holding portions may be used.

The present invention is also directed to a kit, including a lancet device 10 shown by way of example only in FIG. 2A comprising a body portion 11 and a supporting portion for supporting a plurality of spaced sharpened tips 12 in a predetermined pattern with respect to one another, and a collection device holder 20 shown by way of example only in FIG. 2A for a plurality of blood collection tubes 23, the holder 20 comprising a support member 21 and a plurality of holding portions 22, each of the plurality of holding portions 22 being configured to hold a blood collection tube 23 with respect to the support member 21 such that inlets of the plurality of blood collection tubes 23 are arranged in a predetermined pattern with respect to one another corresponding to the a predetermined pattern of the plurality of spaced sharpened tips 23.

While capillary tubes are noted as one example of blood collection vessels that may be used in the present invention, any blood collection vessel may be used. For example, a tube or vessel which is too wide for efficient capillary attraction fill can be used if additional means for filling the tube, such as a suction/plunger are provided, e.g., at a top end of the tube. The Bernoulli effect may also be used to assist in blood collection in a capillary tube by causing air to pass over the top end (opposite the collection inlet) of the tube, e.g., by using a small fan to cause air to pass over the top end. The capillary tube holder unit and the Bernoulli enhancer can readily be configured into a single unit. Enhancement of collection is not limited to Bernoulli-driven suction; any means of creating a pressure differential to increase blood flow and/or collection may be employed.

FIGS. 4A and 4B are schematic views of collection device holders of the present invention having devices for creating a lower pressure region in the collection tubes. As shown in FIG. 4A, a collection device holder 20 is designed for a holding at least one blood collection tube 23, and includes a device, in this embodiment a fan 24, for creating a lower pressure region in the capillary tube 23. In the embodiment shown in FIG. 4A, the fan 24 assists in blood collection in the capillary tube 23 by causing air 25 to pass over the top end (opposite the collection inlet) of the tube 23. The capillary tube holder unit 20 and the collection enhancer can readily be configured into a single unit as shown.

FIG. 4B is schematic views of another embodiment of a collection device holder having a plunger 26 for creating a lower pressure region in the capillary tube 23.

While FIGS. 4A and 4B show only one collection tube, of course the devices for creating a lower pressure region in the collection tubes can be used for collection devices holding multiple tubes.

The capillary tubes can have a dual function of lancet and collection device if the inlet end is sharpened.

Now, while the primary embodiment is for the use of the lancet to take a sample from a finger, it is expressly contemplated by the inventors that the lancet may be used on any geographic region of the patient (for example and without limitation on the arm, leg, etc), or other mammal, including for veterinary use.

In one aspect of the present invention, the lancets may be supported to be held very close to one another so as to form a single blood droplet from the site due to the closeness causing a coalescence of the blood into a droplet. For example, for multiple tube use, since the collection device mimics the geographic placement, the placement is unrestricted in placement other than by practicality. While this invention is for multiple tubes it is also for use of one tube with a larger collection tube using one blood drop made possible by lancets being spaced within an area less than 15 mm diameter (or rectangle having a long side less than 15 mm). Since the first droplet is usually discarded for a lot of test, this makes a second droplet more feasible.

An important goal bifurcation needs to be kept in mind. A multi-lancet device is essential in the following circumstances. Firstly, multiple lancets combined with a holder for multiple capillary type tubes with geographic unity and secondly, multiple lancets, used as to form a larger coalesced blood sample which can be collected by a single larger collection tube whether capillary or suction is employed but the capillary holder is not necessary. Lastly, capillary action filling can be shown to be augmented by the presence of an air current flowing over the top of the capillary tubes. Finally, the presence of an anti clotting type agent in the broadest sense can aid and abet the volume of blood from a lancet piercing

A variety of anti-clotting agents can be used on the lancet (which may be a conventional lancet or the multi-prick invention).

Anti-clotting agents are widely used for various purposes but, to the knowledge of the inventors, have never been used or suggested for the purpose or use of the current invention. They are currently employed, for example, in preventing clots from forming that could lead to a myocardial infarction, stroke, deep vein thrombosis, or pulmonary embolism. They can also be essential with impanted medical devices such as prosthetic heart valves are implanted in the body. Typically, thee drugs target either platelet activation or the blood coagulation reactions.

### Drugs Blocking Platelet Activation

Aspirin blocks thromboxane A2 formation. Other NSAIDs, such as ibuprofen and naproxen, of course do the same thing. But recall that aspirin forms a covalent bond with COX, and thus if a cell is to regain the activity of its COX, it must synthesize more enzyme. And this alters the final result because platelets don't have nuclei, while endothelial cells do. Without a nucleus, they can't synthesize more COX.

Thus, aspirin, because it forms a covalent bond with COX, has a larger effect on platelets than on endothelial cells, which can synthesize more COX due to their nuclei. This is a reason why a comparatively low dose of aspirin has lasting anti-clotting effects.

Clopidogrel (Plavix) is an oral drug used either along with aspirin or as an alternative to aspirin. It inhibits platelet activation by blocking the binding of ADP to its receptor. Both aspirin and clopidogrel are most commonly used to reduce the probability of clot formation in blood vessels damaged by atherosclerosis. Thus they are widely used in patients at risk of a myocardial infarction.

Dipyridamole has several effects that reduce activation of platelets. For example, it inhibits the uptake of adenosine by platelets, which increases the extracellular concentration of adenosine. Adenosine blocks the effect of ADP. (Incidentally, adenosine forms in the blood due to an enzyme that acts on ADP. Thus, more ADP in the blood leads to the formation of adenosine, a blocker of ADP.)

Abciximab(Reopro) is a monoclonal antibody that binds to the glycoprotein IIb/IIIa receptor, thereby interrupting platelet aggregation. Since abciximab is a protein it must be injected. Thus, it is used during invasive medical procedures that might trigger clot formation, such as angioplasty for opening narrowed coronary arteries. There are some other drugs in this category too.

### Drugs Blocking the Blood Coagulation Reactions

Warfarin (Coumadin) is a widely used anticoagulant drug. It acts as a vitamin K antagonist. Since vitamin K is required for the activity of prothrombin as well as several other blood coagulation proteins, warfarin decreases the formation of thrombin and thus fibrin. A patient on warfarin requires regular blood tests since warfarin has narrow therapeutic range; that is, its effect is quite sensitive to its exact concentration. The tests measure how fast the blood coagulates. One of the most common uses is in atrial fibrillation, in which stagnant blood in the atria of the heart tends to lead to the formation of clots. This is a very common condition in the elderly. But it is used in various other situations as well, such as deep vein thrombosis and implantation of artificial prosthetic devices such as heart valves.

Direct Inhibitors of Thrombin and Factor Xa are relatively new drugs and are an alternative to warfarin. While warfarin blocks the vitamin K necessary for their activity, these drugs block the active substances directly. One difference between warfarin and these drugs is that the effects of warfarin can be quickly reversed. But the direct inhibitors continue working even if the factors are increased. However, recent clinical trials for the most part have shown that with atrial fibrillation the direct inhibitors ultimately are as safe and effective as warfarin, if not more so. Moreover, they do not require the constant blood testing. One example is dabigatran (Pradaxa), which is a thrombin inhibitor. Another is apixaban (Eliquis), which blocks factor Xa.

Heparin is another commonly used anticoagulant. It has the same effect as endothelial heparan proteoglycan in activating anti-thrombin, although what the effect of heparin itself in the body is not clear. (It is released from mast cells.) Heparin is purified from sources such as pig intestine. The purified product contains many different chain lengths, and this is the form in which it is often used. But sometimes a further purified form called low molecular weight heparin (enoxaparin, etc) is used. The idea behind this drug is that the more refined product with only the smaller sized molecules provides a more measured effect (since it has a bigger effect on a blood coagulation factor than on prothrombin itself). Heparin is used for many of the same problems as warfarin. However, since it must be injected, it is used when a short term, rapid effect is desired. Heparin is also used to coat test tubes used in collecting blood, intravenous catheters, and similar devices.

Drugs that Break Down Clots

The first drug used for this purpose is tissue plasminogen activator (tPA). This is an enzyme that normally converts plasminogen to plasmin in the blood. Plasmin is an enzyme that breaks down fibrin. Clots are temporary structures in the body, and plasmin is the normal enzyme that starts the destruction of a clot after it forms. Since it is a protein, tPA must be injected. Recombinant DNA technology is used to manufactor tPA.

Another option is streptokinase, which is a less expensive enzyme purified from bacteria.

The inventors have described the anti-clotting agents above merely as representative agents that can be used as anti-clotting agents on the lancets according to the present invention. The inventors expressly contemplate that any anti-clotting agent (currently known or developed in the future) could be used in connection with the present invention.

Now, it should be noted that heparin has been used to coat test tubes, as well as the inside of the barrel of syringe needles used to draw arterial blood (e.g. for blood gas). Industry leader Becton Dickinson has described such in products in WO 2013059438 A1 (“Syringe with breakable plunger for arterial blood gas sample collection”); CA 2466506 C (“Spray dry process for applying anticoagulant on a syringe barrel”); and C 2501968 C (“Flush syringe having anti-reflux features”). Each of these references is hereby incorporated by reference as if fully set forth herein (together with their references). However, none of this art teaches or suggests the present invention, being directed to coating different devices (syringe barrels and test tubes) for different reasons (i.e. clot prevention in the syringe barrel or tube).

It is contemplated that sufficient clotting agent is used to increase blood draw, but without sufficient dose to provoke systemic effects.

The lancet may employ one or more anti-clotting agents (i.e. a combination of agents) together.

The anti-clotting agent can be adhered to, or coated on or in the lancet using any known coating method. It is also contemplated that the anti-clotting agent may be applied immediately prior to the use of the lancet.

The present invention can be used to collect blood, e.g., venous blood, punctures in skin of a mammal using a lancet device of the present invention. After a droplet has formed, the blood is sucked up in a capillary tube or a plurality of capillary tubes, relying on surface tension, or, if desired, using indirect suction. For example, the method for collecting blood can include producing multiple punctures in skin of a mammal using a lancet device having a body portion and a supporting portion supporting a plurality of spaced sharpened tips in a predetermined pattern with respect to one another; providing a collection device holder for a plurality of blood collection tubes, the holder including a support member and a plurality of holding portions, each of the plurality of holding portions holding a blood collection tube with respect to the support member such that inlets of the plurality of blood collection tubes are arranged in a predetermined pattern with respect to one another corresponding to the a predetermined pattern of the plurality of spaced sharpened tips; and collecting blood from the multiple punctures by touching the inlets of the plurality of blood collection tubes to the multiple punctures.

### Example 1

Capillary tubes made by Kimble Chase, i.e., Plain Capillary Tubes I.D. 1.1-1.2 mm Wall 0.2+ or −0.02 mm, Length 75 mm and Heparinized Capillary Tubes ID 1.1-1.2 mm Wall 0.2+ or minus 0.02 mm, Length 75 mm were used to collect water with blue food coloring. Using a small hair dryer, the capillary tubes were used to gather the colored water at an angle by capillary action alone. They filled to approximately ½ of the tube. Then air was blown gently but steadily over the top are of the tubes. The result was the tubes filled to approximately ⅔ of the tube. If air was stopped the tubes dropped back to ½ full. The results indicate the Bernoulli effect works quite well to increase liquid collection. Therefore, it was concluded that if a capillary tube is used to obtain blood it will be more effective if an air field passes over the top end(s) of the tube(s) regardless of the position of the top end(s) vertically.

Naturally any use of methods such as applying warmth, dependency etc. that promote capillary dilation may well be helpful. In addition, if cold is precycled with warmth it can help to first drain the capillary field and then, immediately or within a few (e.g., within 5, preferably within 2, most preferably within 1) minutes when warmth is applied the capillary field is filled with fresher blood. The puncture and collection should then be done immediately or within 5 minutes.

### Example 2

This example uses a glucose meter to demonstrate that cold is precycled with warmth it can help to first drain the capillary field and then when warmth is applied the capillary field is filled with fresher blood. In this example, for the same individual, a Freestyle blood glucose unit, after 2 minutes of ice cube contact followed by 4 minutes of warmth, consistently showed a different result in the same patient using the same finger, when compared to the cold cycle being omitted. More specifically, on the first of two successive days, cold (by applying ice to the individual's finger) was applied for 2 minutes and then (immediately thereafter) warmth was applied (microwavable heat pad around finger) for 4 minutes. After this procedure, the reading Freestyle blood glucose unit was 124. After waiting 30 minutes and applying warmth only, the reading was 118. On the second of two consecutive days, the same procedure was conducted and the reading for cold followed by warmth was 128 and for warmth only was 121.

1. A collection device holder for a plurality of blood collection tubes, the holder comprising a support and a plurality of holding portions, each of the plurality of holding portions being configured to hold a blood collection tube from the plurality of blood collection tubes with respect to the support such that inlets of the plurality of blood collection tubes are arranged in a predetermined pattern with respect to one another and such that inlets of the plurality of blood collection tubes can be arranged in more than one plane.

2. The collection device holder according to claim 1, wherein the predetermined pattern geographically corresponds to prick sites from a multiple prick lancet device.

3. (canceled)

4. The collection device holder according to claim 1, further comprising plurality of blood collection tubes, each of the plurality of blood collection tubes being held in each of the plurality of holding portions such that inlets of the plurality of blood collection tubes are arranged in more than one plane.

5. The collection device holder according to claim 1, wherein each of the plurality of holding portions is configured to hold a blood collection tube from the plurality of blood collection tubes such that the blood collection tube is movable in a longitudinal direction of the blood collection tube so as to enable relative depth adjustment of the inlets of the plurality of blood collection tubes.

6. The collection device holder according to claim 1, further comprising a device for creating a lower pressure region in the capillary tube.

7. A collection device holder for a holding at least one blood collection tube, comprising a holder comprising a support member and a at least one holding portion configured to hold at least one blood collection tube, and a device for creating a lower pressure region in the capillary tube.

8-12. (canceled)

13. A kit comprising:
a lancet device comprising a body portion and a supporting portion for supporting a plurality of spaced sharpened tips in a predetermined pattern with respect to one another; and
a collection device holder, separate from the lancet device, for a plurality of blood collection tubes, the holder comprising a support and a plurality of holding portions, each of the plurality of holding portions being configured to hold a blood collection tube from the plurality of blood collection tubes with respect to the support such that inlets of the plurality of blood collection tubes are arranged in a predetermined pattern with respect to one another corresponding to the predetermined pattern of the plurality of spaced sharpened tips.

14. (canceled)

15. The kit according to claim 13, wherein each of the plurality of sharpened tips has an anti-clotting agent provided thereon.

16. (canceled)

17. A method for collecting blood, comprising:
producing multiple punctures in skin of a mammal using a lancet device comprising a body portion and a supporting portion supporting a plurality of spaced sharpened tips in a predetermined pattern with respect to one another;
providing a collection device holder for a plurality of blood collection tubes, the holder comprising a support member and a plurality of holding portions, each of the plurality of holding portions holding a blood collection tube with respect to the support member such that inlets of the plurality of blood collection tubes are arranged in a predetermined pattern with respect to one another corresponding to the a predetermined pattern of the plurality of spaced sharpened tips; and
collecting blood from the multiple punctures by touching the inlets of the plurality of blood collection tubes to the multiple punctures.

18. A method for collecting blood using cold to warm cycling to refresh the capillary field prior to lancet use, comprising:
cooling a portion of the skin of a mammal;
then, within 60 seconds after the cooling, warming the portion of the skin;
then, within 5 minutes after the warming, producing at least one puncture in skin of a mammal; and
collecting blood from the at least one puncture.

19. A method for collecting blood, comprising:
producing at least one puncture in skin of a mammal; and
collecting blood from the at least one puncture in a collection tube open at each end while creating a pressure differential over the end of the collection tube opposite the collection end so that blood is more easily drawn into the collection tube.

20. The kit according to claim 13, wherein each of the plurality of holding portions is configured to hold a blood collection tube from the plurality of blood collection tubes such that inlets of the plurality of blood collection tubes are arranged in more than one plane.

21. The kit according to claim 13, wherein each of the plurality of holding portions is configured to hold a blood collection tube from the plurality of blood collection tubes such that the blood collection tube is movable in a longitudinal direction of the blood collection tube so as to enable relative depth adjustment of the inlets of the plurality of blood collection tubes.
